(19) United States
(12) Patent Application Publication
(10) Pub. No.: US 2025/0222154 A1
(43) Pub. Date: Jul. 10, 2025

(54) MAGNETIC FRAGRANCE CARD

(71) Applicant: Hua Ting WU, Taipei City (TW)

(72) Inventor: Hua Ting WU, Taipei City (TW)

(21) Appl. No.: 18/614,885

(22) Filed: Mar. 25, 2024

(51) Int. Cl. A61L 9/12; H04M 1/02

(52) U.S. Cl. CPC A61L 9/12; H04M 1/0279; A61L 2209/15

(57) ABSTRACT

A magnetic fragrance card configured to be mounted on an electronic device includes a card body, a magnetic element, an embedded element, and a fragrance element. The card body has a first surface and a second surface, a mounting groove, and a through hole, the mounting groove formed on the first surface, and the through hole penetrating the first surface and the second surface. The magnetic element is mounted in the mounting groove; the embedded element, having an accommodation space, is mounted in the through hole, and an opening end of the accommodation space is at the same side of the second surface; the fragrance element is mounted in the accommodation space; wherein the first surface of the card body is magnetically attached to the electronic device via the magnetic element.

### CROSS-REFERENCE TO RELATED APPLICATIONS

This application claims the priority benefit of TW application serial No. 113200198, filed on Jan. 5, 2024. The entirety of the above-mentioned patent application is hereby incorporated by reference herein and made a part of the specification.

### BACKGROUND OF THE INVENTION

### 1. Field of the Invention

The present invention relates to a fragrance card, and more particularly to a magnetic fragrance card.

### 2. Description of the Related Art

Fragrances can improve environmental odors, enhance air quality, affect brain and body functions through the sense of smell, relieve tension and anxiety, aid sleep, and increase concentration. Fragrance products are increasingly becoming popular and important, and with the development of fragrance products, application of fragrance product is no longer limited to indoor environments but can be applied to portable electronic devices such as smartphones.

Currently, a fragrance device applied to mobile phones is attached to the metal casing of the phone through magnetic adsorption. Such a fragrance device generally includes an upper cover, a lower cover, a magnetic element, and a fragrance element. The fragrance element is placed between the upper and lower covers, encapsulated by them, while the magnetic element is located in an accommodation slot on the lower surface of the lower cover, allowing the fragrance device to adhere to the phone via the magnetic element. Furthermore, the lower cover has at least one slot exposing the fragrance element, so that the user can refill essential oil to the fragrance unit through at least one slot conveniently, ensuring the functionality of the fragrance device.

To avoid the slot affecting the aesthetic appearance of the fragrance device, the slot is typically located on the lower surface of the lower cover. When the fragrance device is attached to the mobile phone via the magnetic element on the lower surface of the lower cover, the slot is concealed between the lower cover and the casing of the mobile phone. However, since the fragrance element is exposed through the slot, there is no barrier between the fragrance element and the mobile phone at the slot. Therefore, the essential oil filled in the fragrance unit and the fragrance vapors emitted by the fragrance element may adhere to the phone's casing through the slot, which may cause discoloration, peeling-off of paint or coating of the casing, leading to user inconvenience.

On the other hand, as the fragrance element and the magnetic element are respectively located on the upper and lower sides of the lower cover, the lower cover requires different accommodation spaces to house the fragrance element and the magnetic element separately, resulting in a larger volume of the fragrance device and difficulty in reducing its thickness, which is a disadvantage in slimming of the fragrance device. Additionally, when the fragrance device is mounted on the phone, the bulky volume may affect the feel of the user when operating the phone.

Therefore, the current fragrance devices need to be further improved.

### SUMMARY OF THE INVENTION

An objective of the present invention is to provide a magnetic fragrance card. The magnetic fragrance card disposes a magnetic element and a fragrance element through the card body, reducing the thickness of the magnetic fragrance card, providing an advantage for slimness of the magnetic fragrance card. The fragrance element is mounted in the embedded element; therefore, the fragrance element is separated from the electronic device, preventing the fragrance component in the fragrance element from interfering with the appearance and usage of the electronic device.

To achieve the foregoing objective, the magnetic fragrance card includes:

card body, having a first surface, a second surface opposite to the first surface, a mounting groove, and a through hole, the mounting groove formed on the first surface, and the through hole penetrating the first surface and the second surface;

a magnetic element, mounted in the mounting groove;

an embedded element, having an accommodation space and mounted in the through hole, and an opening end of the accommodation space located at a side of the second surface;

a fragrance element, mounted in the accommodation space; wherein

the first surface of the card body is magnetically attached to the electronic device via the magnetic element.

The magnetic element is installed on the first surface of the card body through the mounting groove, the embedded element is installed in the through hole of the card body, and the fragrance element is located in the accommodating space of the embedded element. Both the magnetic element and the fragrance element are mounted in the card body, reducing the volume and thickness of the magnetic fragrance card, aiding in slimming of the magnetic fragrance card. Moreover, the fragrance element is located within the accommodation space of the embedded element, and the opening end of the accommodation space is at the same side of the card body with the second surface. This prevents the fragrance components emitted by the fragrance element from contacting the surface of the electronic device by separating the fragrance element and the electronic device, thus avoiding damage to the electronic device.

Other objectives, advantages and novel features of the invention will become more apparent from the following detailed description when taken in conjunction with the accompanying drawings.

### BRIEF DESCRIPTION OF THE DRAWINGS

FIG. 1 is a three-dimensional schematic diagram of the magnetic fragrance card in a first embodiment of the present invention.

FIG. 2 is another three-dimensional schematic diagram of the magnetic fragrance card in the first embodiment of the present invention.

FIG. 3 is a sectional schematic diagram of the magnetic fragrance card in the first embodiment of the present invention.

FIG. 4 is a schematic diagram of the magnetic fragrance card mounted on a mobile phone in the first embodiment of the present invention.

FIG. 5 is another schematic diagram of the magnetic fragrance card mounted on a mobile phone in the first embodiment of the present invention.

FIG. 6 is a three-dimensional schematic diagram of the magnetic fragrance card in a second embodiment of the present invention.

FIG. 7 is another three-dimensional schematic diagram of the magnetic fragrance card in the second embodiment of the present invention.

FIG. 8 is a sectional schematic diagram of the magnetic fragrance card in the second embodiment of the present invention.

FIG. 9 is a schematic diagram of the magnetic fragrance card mounted on a mobile phone in the second embodiment of the present invention.

FIG. 10 is another schematic diagram of the magnetic fragrance card mounted on a mobile phone in the second embodiment of the present invention.

### DETAILED DESCRIPTION OF THE INVENTION

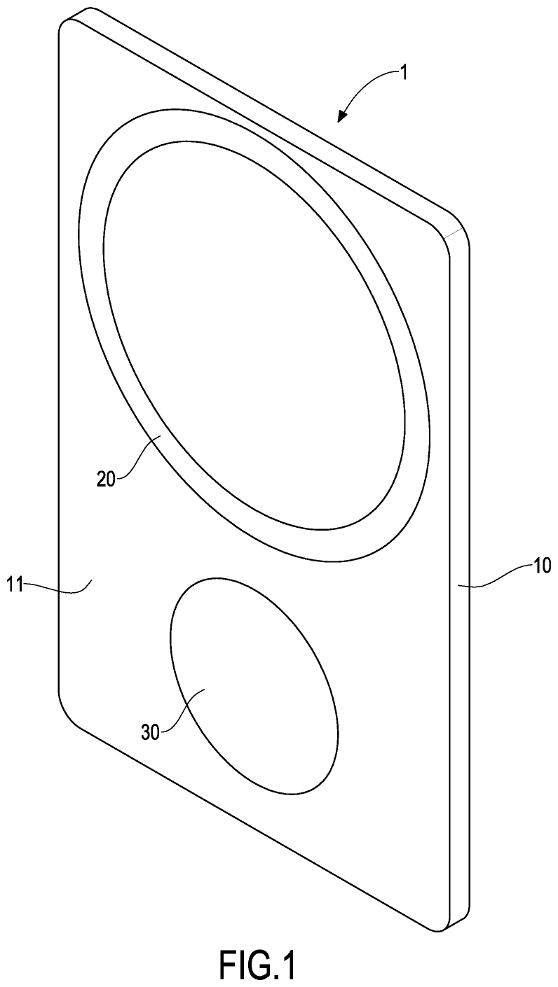

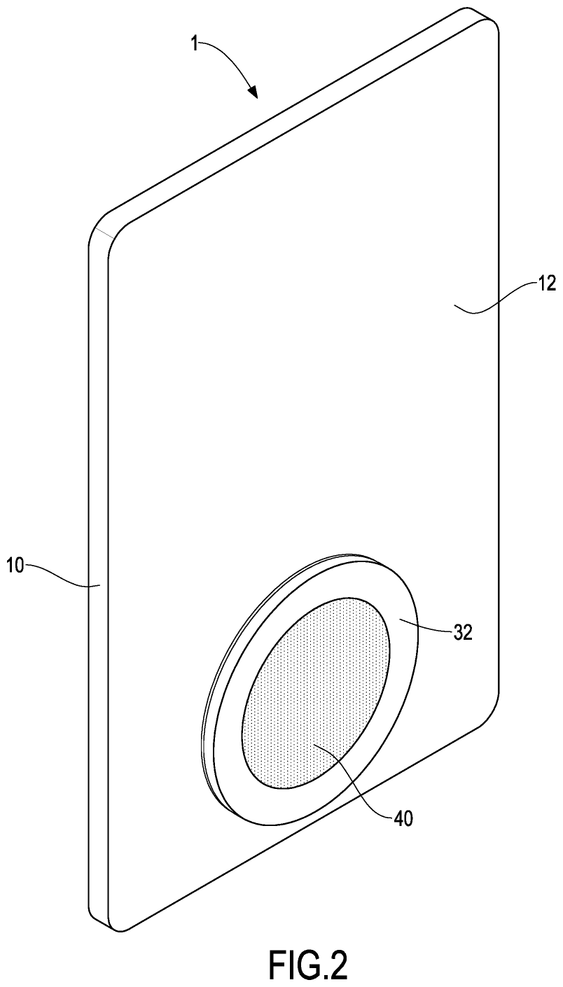

With reference to FIG. 1 to FIG. 3, wherein the present invention is a magnetic fragrance card 1, including a card body 10, a magnetic element 20, an embedded element 30 and a fragrance element 40.

The card body 10 has a first surface 11, a second surface 12, a mounting groove 13, and a through hole 14. The first surface 11 is opposite to the second surface 12.

The mounting groove 13 is located on the first surface 11, and the through hole 14 penetrates the first surface 11 and the second surface 12. In the first embodiment, the card body 10 is a rectangular sheet.

The magnetic element 20 is mounted in the mounting groove 13 of the card body 10. Wherein the magnetic element 20 may be attached to the mounting groove 13 by adhesive glue or heat fusion. In another embodiment, the magnetic element 20 may also be fixed in the mounting groove 13 by screw thread structures or latch structures.

In a preferred embodiment, the mounting groove 13 is an annular mounting groove 13, and the magnetic element 20 is an annular magnetic element 20. The magnetic element 20 is mounted in the mounting groove 13, and the surface of the magnetic element 20 is coplanar with the first surface 11.

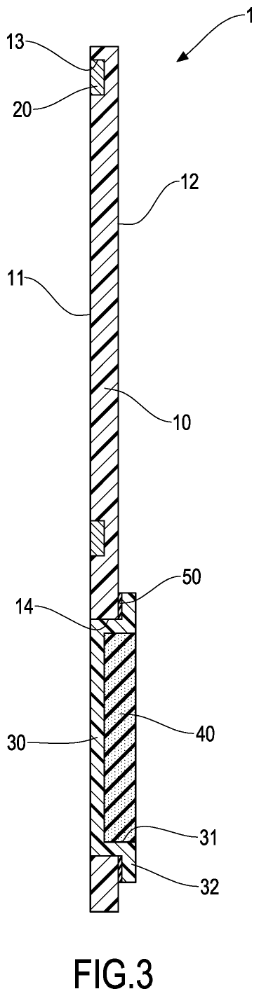

The embedded element 30 has an accommodation space 31 and a fixing part 32. The accommodation space 31 has an opening end, and the fixing part 32 is located at this opening end of the accommodation space 31. The embedded element 30 is inserted through the through hole 14 of the card body 10, and the fixing part 32 of the embedded element 30 connects to the second surface 12 of the card body 10. The opening end of the accommodation space 31 and the second surface 12 are on the same side. The fixing part 32 can be connected to the second surface 12 using an adhesive or by heat fusion. In FIG. 3, the fixing part 32 is attached to the second surface 12 using adhesive glue 50.

The fragrance element 40 is positioned within the accommodation space 31 of the embedded element 30, where it can be attached using an adhesive or by heat fusion. The fragrance element 40 can also be secured within the accommodation space 31 using a snap-fit structure (not shown in the figure), and can be made from at least one material capable of adsorbing fragrance oils, such as paper, bamboo, rattan, plaster, ceramic, diatomaceous earth, wood, fabric, lava, or crystal, combined with fragrance oils. The fragrance element 40 can be replenished with fragrance oils to restore its fragrance function and continuously emit scent.

In a preferred embodiment, the opening end of the accommodation space 31 is equipped with a cover body that has ventilation holes. The cover body is placed over the opening end to prevent the fragrance element 40 from being directly exposed. The embedded element 30 is made of a metallic material, and the cover body can be a magnetic cover, which is attached to the opening end of the accommodation space 31 of the embedded element 30 via magnetic attraction.

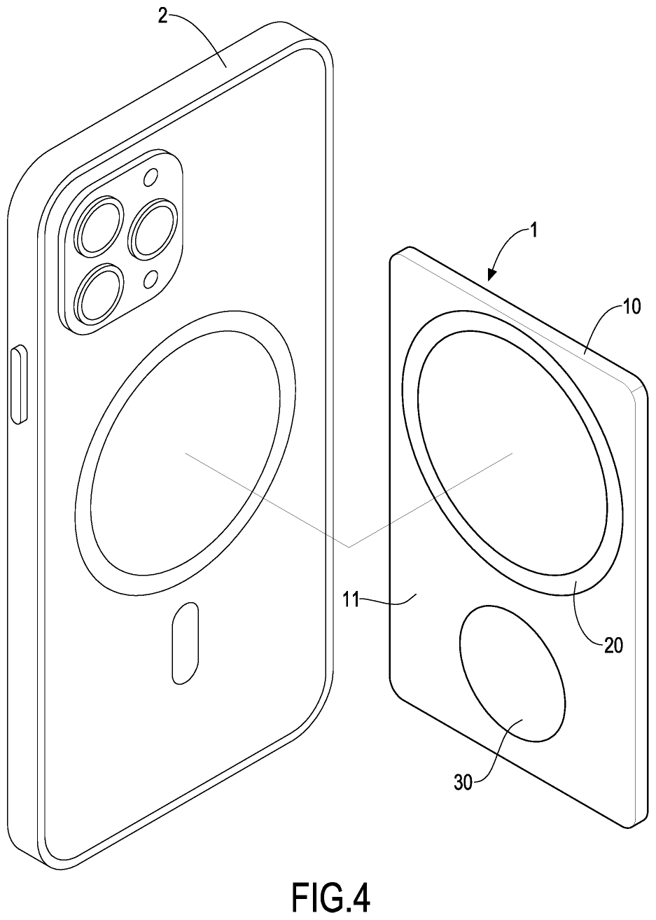

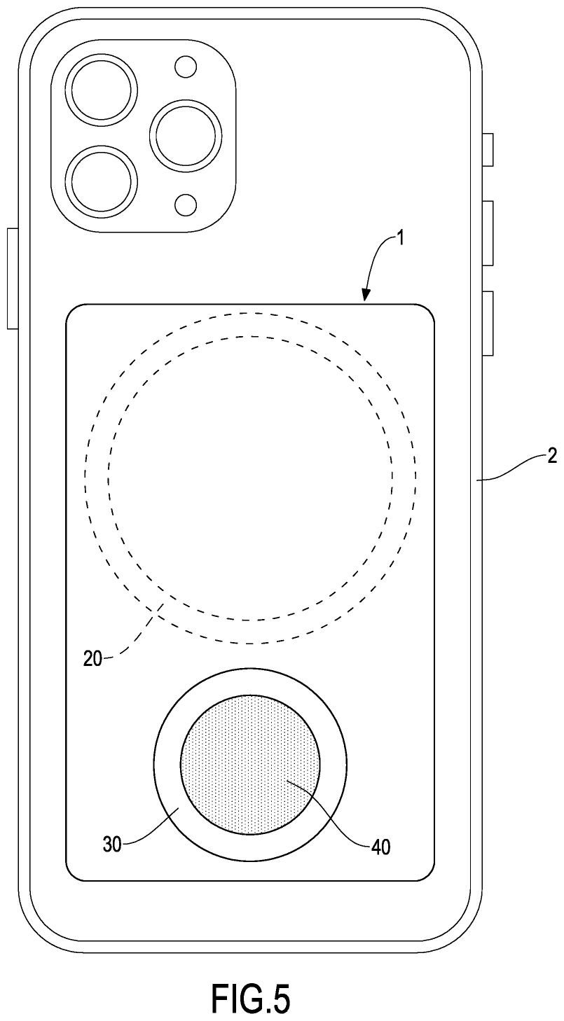

With reference to FIGS. 4 and 5, the magnetic fragrance card 1 can be attached to an electronic device 2 to provide fragrance functionality. The magnetic fragrance card 1 is attached to the electronic device 2 by magnetic force attraction by its first surface 11, wherein the electronic device 2 may include portable electronic devices such as a mobile phone or a tablet computer. In FIGS. 4 and 5, the electronic device 2 is a mobile phone, and the magnetic fragrance card 1 is attached to the mobile phone via the magnetic element 20, connecting the first surface 11 to the surface of the phone case.

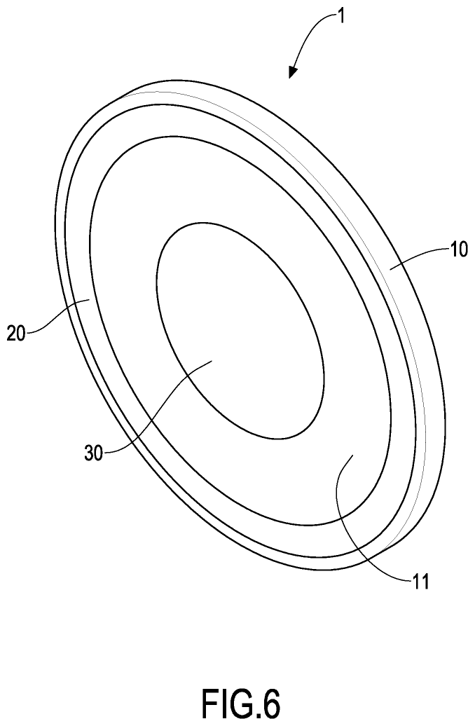

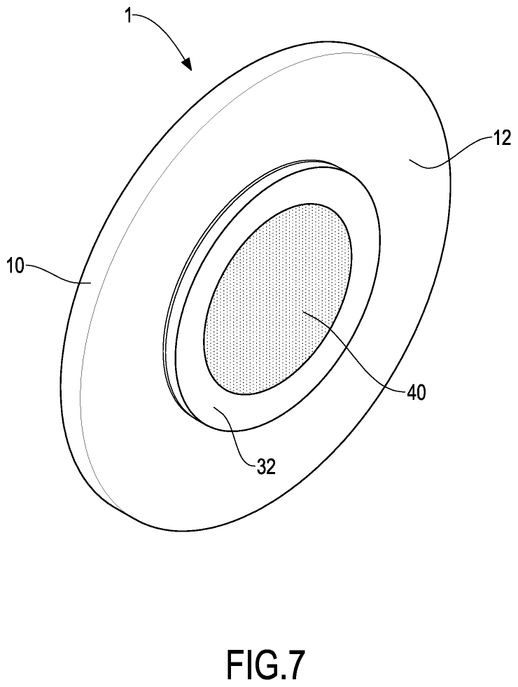

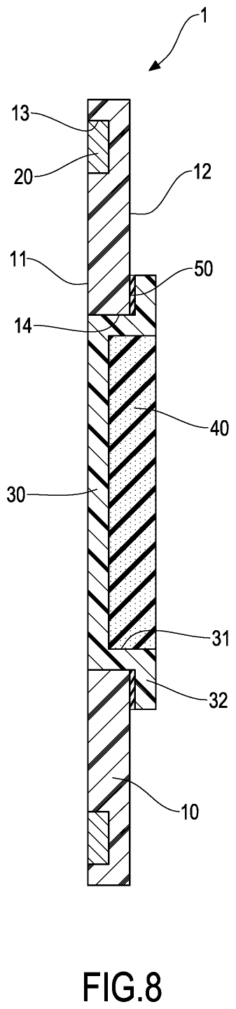

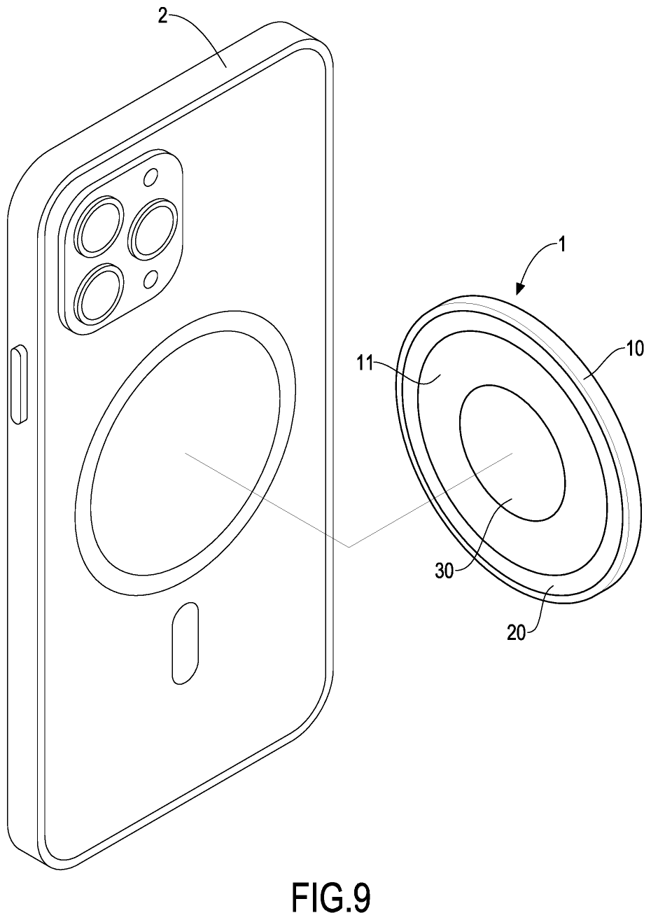

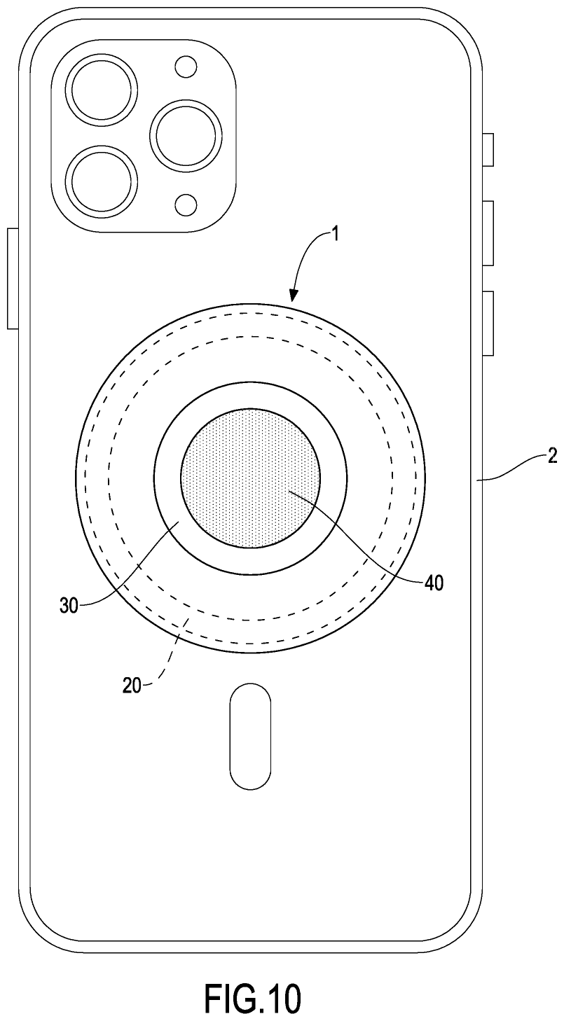

With reference to FIG. 6 to FIG. 10, the difference between the second embodiment and the first embodiment is that, in the second embodiment, the card body 10 is a circular disc, and the through hole 14 is located at the inner side of the mounting groove 13, meaning the mounting groove 13 encircles the outer perimeter of the through hole 14. In FIG. 6 to FIG. 10, the mounting groove 13 is exemplified as an annular mounting groove, with the through hole 14 located at the inner side of this annular mounting groove, i.e., the annular mounting groove 13 is located around the outer perimeter of the through hole 14.

In the present invention, the fragrance element 40 is positioned within the accommodation space 31 of the embedded element 30, and the opening end of the accommodation space 31 and the second surface 12 of the card body 10 are on the same side. When the magnetic fragrance card 1 is mounted on the electronic device 2 by the first surface 11 of the card body 10, the embedded element 30 accommodates the fragrance element 40, keeping it separated from the electronic device 2, preventing the fragrance gas or components emitted by the fragrance element 40 from directly contacting the surface of the electronic device 2, thereby avoiding damage to the device such as discoloration or paint peeling-off of the casing.

Moreover, with the magnetic element 20 positioned on the first surface 11 of the card body 10, the embedded element 30 is inserted in the through hole 14 of the card body 10, and the fragrance element 40 is mounted in the accommodation space 31 of the embedded card body 10. This configuration reduces the volume and thickness of the magnetic fragrance card 1, contributing to the slimness of the magnetic fragrance card 1.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A magnetic fragrance card, configured to be mounted on an electronic device, the magnetic fragrance card comprising:
a card body, having a first surface, a second surface opposite to the first surface, a mounting groove, and a through hole, the mounting groove formed on the first surface, and the through hole penetrating the first surface and the second surface;
a magnetic element, mounted in the mounting groove;
an embedded element, having an accommodation space and mounted in the through hole, and an opening end of the accommodation space located at a side of the second surface;
a fragrance element, mounted in the accommodation space; wherein
the first surface of the card body is magnetically attached to the electronic device via the magnetic element.

2. The magnetic fragrance card as claimed in claim 1, wherein the embedded element has a fixing part, and the fixing part is mounted on the opening end of the accommodation space and attached to the second surface of the card body.

3. The magnetic fragrance card as claimed in claim 2, wherein the fixing part is attached to the second surface of the card body by an adhesive glue.

4. The magnetic fragrance card as claimed in claim 1, wherein the through hole is located at an inner side of the mounting groove.

5. The magnetic fragrance card as claimed in claim 1, wherein the mounting groove is an annular mounting groove, and the through hole is located at an inner side of the annular mounting groove.

6. The magnetic fragrance card as claimed in claim 1, wherein a surface of the magnetic element is coplanar with the first surface.

7. The magnetic fragrance card as claimed in claim 1, further comprising a cover body covering the opening end of the accommodation space.

8. The magnetic fragrance card as claimed in claim 7, wherein the cover body is a magnetic cover body.

9. The magnetic fragrance card as claimed in claim 1, wherein the card body is one of the followings:
a rectangular card body and a circular card body.
